(19) United States
(12) Patent Application Publication
(10) Pub. No.: US 2022/0015688 A1
(43) Pub. Date: Jan. 20, 2022

(54) SWEAT SENSORS BASED ON MEASURING A SWELLABLE VOLUME

(71) Applicant: University Of Cincinnati, Cincinnati, OH (US)

(72) Inventors: Mikel Larson, Cincinnati, OH (US); Mathias Bonmarin, Cincinnati, OH (US); Jason Charles Heikenfeld, Cincinnati, OH (US)

(21) Appl. No.: 17/295,091

(22) PCT Filed: Nov. 20, 2019
(86) PCT No.: PCT/US2019/062418

§ 371 Date: May 19, 2021

Related U.S. Application Data
(60) Provisional application No. 62/769,763, filed on Nov. 20, 2018.
(60) Provisional application No. 62/835,261, filed on Apr. 17, 2019.

(51) Int. Cl. A61B 5/00; A61B 10/00; A61B 5/1455; A61B 5/145; A61B 5/1468; A61B 5/1495

(52) U.S. Cl. CPC A61B 5/4266; A61B 10/0064; A61B 5/681; A61B 5/1455; A61B 2562/0271; A61B 5/14546; A61B 5/1468; A61B 5/1495; A61B 5/14517

(57) ABSTRACT

A sweat sensing device is described. The sweat sensing device includes at least one swellable component. The sweat sensing device further includes a defined sweat collection area in fluid communication with the swellable component. The sweat sensing device further includes at least one first sensor for directly or indirectly measuring the dimension of the swellable component such that sweat generation rate and/or sweat volume can be calculated from the measure of dimension of the swellable component and the defined sweat collection area.

### CROSS-REFERENCE TO RELATED APPLICATIONS

This application claims priority to, and the benefit of the filing date of, U.S. Provisional Application No. 62/835,261 filed Apr. 17, 2019, the disclosure of which is hereby incorporated by reference in its entirety. This application further claims priority to, and the benefit of the filing date of, U.S. Provisional Application No. 62/769,763 filed Nov. 20, 2018 the disclosure of which is hereby incorporated by reference herein in its entirety.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Systems and devices that measure exposure to internal and external fluids are crucial for various applications. Internal exposure refers to exposure of sensors to an internal fluid source of a subject's body. An example of this is sweat sensing for assessment of body hydration. External exposure refers to exposure of sensors to an external or environmental fluid source not coming from a subject's body. An example of this is the measurement of exposure to a potentially toxic substance like acetone, ethanol, hexane, or methanol.

Numerous devices have been proposed or developed and demonstrated for measuring exposure to internal fluids and/or external fluids (for example, see S. A. Kolpakov et al. Toward a New Generation of Photonics Humidity Sensors, Sensors 2014, 14, 3986-4013.). These devices can be separated into two types: (1) sensors that provide an accurate measurement of fluid content, and (2) indicators that give an imprecise indication about fluid content. For example, numerous sweat rate and sweat volume sensors have been proposed or developed and demonstrated for use in monitoring hydration loss or for use in monitoring sweat rate to inform chronological assurance and/or to inform analyte dilution with increased sweat generation rate.

Regarding the first type of device (i.e., sensors): Many sensors rely on electrical methods of measuring fluid exposure. For example, such sensors may measure conductivity or resistivity of a fluid (see International Patent Publication Nos. WO 2015/05855 and WO 2016/007944). However, such sensors are problematic because fluid conductivity or resistivity is dependent on various parameters, which can change from sample to sample, thereby limiting the use of the sensor in particular when measuring biological fluids like sweat, blood, tears, etc. For example, the above-mentioned sensors cannot be used to accurately measure sweat, as salinity of this fluid dramatically varies from one subject to another, and with sweat rate.

Other sensors rely on methods that measure a position of a moving fluid in a channel, which is more reliable, but which typically requires a positive pressure to drive sweat into the channel, requiring placement on skin with significant pressure and fixturing to ensure a proper seal against skin. Disposable units including sensors using such methods are costly. Thus, there is a need for improved methods to measure sweat generation rate that are lower cost, that do not rely on methods such as measurement of conductivity or position of a moving fluid, and that are highly accurate.

Regarding the second type of device (i.e., indicators): In most of these devices, a fluid-sensitive chemical is impregnated on a surface such that it will change color or appearance when the indicated relative fluid quantity is exceeded. These indicators are very popular for humidity measurements (see U.S. Patent Application Publication No. 2018/356379). Such indicators typically include blotting paper impregnated with cobalt chloride base or other less toxic alternatives. Similar methods have been used for diapers to indicate the amount of urine (see International Patent Publication No. WO 2009/133731) or to measure the amount of sweat produced (see International Patent Publication No. WO 2019/023195 and U.S. Patent Application Publication No. 2018/249952). However, these indicators are problematic because they use fluid-specific chemical reactions to produce color or appearance changes. As a result, it can be the case that no chemical reactions exist for a specific fluid to be measured, or that the reactants are highly toxic for humans or the environment, or that the reactants are expensive.

Recently, a hydrogel interferometer coupled with smartphone-based sensing has been proposed (See M. Qin et al., Bioinspired Hydrogel Interferometer for Adaptive Coloration and Chemical Sensing, Advanced Materials, 2018, 30, 1300468). This sensor is based on a single layer of hydrogel that swells and expands when in contact with a fluid, creating interference patterns. However, such a device is problematic as the interference pattern is extremely wavelength-sensitive and angle-sensitive, resulting in large inaccuracies in the sensor reading under various light conditions. In addition, as the device is very sensitive to hydrogel thickness variations, the indicator might exhibit spatial inhomogeneities, resulting in large inaccuracies in the sensor reading. Finally, the substrate used is not permeable to most fluids and so the sensor cannot be used for the measurement of internal fluid exposure.

In view of the above, there is a need for improved methods to measure both internal and external fluid exposure that are versatile, low cost, accurate, and environment friendly.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

And so, one aspect of the disclosed invention is directed to use of a sweat rate sensor that wicks sweat into a swellable volume, and detects or measures a physical dimension or other characteristic of that swellable volume vs. time to calculate a measure of sweat rate or sweat volume.

Thus, in one general embodiment, a sweat sensing device is described. The sweat sensing device includes at least one swellable component. The sweat sensing device further includes a defined sweat collection area in fluid communication with the swellable component. The sweat sensing device further includes at least one first sensor for directly or indirectly detecting or measuring a dimension or other characteristic of the swellable component such that sweat generation rate and/or sweat volume can be calculated from the measure of dimension of the swellable component and the defined sweat collection area.

Another aspect of the disclosed invention is directed to a method of calculating a sweat volume and/or a sweat rate. The method includes absorbing an amount of sweat from skin into a device. The device includes a swellable component, a defined sweat collection area in fluid communication with the swellable component, and at least one sensor. The method further includes detecting or measuring a dimension or other characteristic of the swellable component with the at least one sensor such that a sweat generation rate and/or a sweat volume can be calculated from the dimension or other characteristic of the swellable component.

### BRIEF DESCRIPTION OF THE DRAWINGS

The objects and advantages of the disclosed invention will be further appreciated in light of the following detailed descriptions and drawings in which:

FIG. 1A is a cross-sectional view of a device in accordance with principles of the present invention with little or no sweat collected.

FIG. 1B is a cross-sectional view of a device in accordance with principles of the present invention with greater sweat collected than the device of FIG. 1A.

FIG. 2A is a cross-sectional view of a device in accordance with principles of the present invention with an optically measured swellable component.

FIG. 2B is a cross-sectional view of a portion of an example of a device of FIG. 2A with no absorption of sweat.

FIG. 2C is a cross-sectional view of a portion of an example of a device of FIG. 2A with absorption of sweat.

FIG. 2D is a cross-sectional view of a portion of an example of a device of FIG. 2A with no absorption of sweat.

FIG. 2E is a cross-sectional view of a portion of an example of a device of FIG. 2A with absorption of sweat.

FIG. 3 is a cross-sectional view of a device in accordance with principles of the present invention with an optically measured swellable component.

FIG. 4A is a cross-sectional view of a device in accordance with principles of the present invention with a swellable component.

FIG. 4B is a top-down view of a device in accordance with principles of the present invention with a swellable component.

FIG. 4C is a top-down view of a device in accordance with principles of the present invention with a swellable component.

FIG. 4D is a top-down view of a device in accordance with principles of the present invention with a swellable component.

FIG. 5A is a cross-sectional view of a device in accordance with principles of the present invention with a swellable component.

FIG. 5B is a cross-sectional view of a device in accordance with principles of the present invention with a swellable component.

FIG. 6A is a cross-sectional view of a device in accordance with principles of the present invention based on measuring geometry of at least one swellable component.

FIG. 6B is a layout of a plurality of swellable components to be used in an example device in accordance with principles of the present invention based on measuring geometry of at least one swellable component.

FIG. 6C is a view of an example of a use of a mobile device reading an example device in accordance with principles of the present invention.

FIG. 7A is a cross-sectional view of a device in accordance with principles of the present invention based on measuring optical reflectance.

FIG. 7B is a view of an example of a use of a mobile device reading an example device in accordance with principles of the present invention.

FIG. 7C is a view of a plurality of sensors that are included in an embodiment in accordance with principles of the present invention.

FIG. 8A is a perspective view of an example of a device in accordance with principles of the present invention.

FIG. 8B is a plot of data collected from a demonstration of a device in accordance with principles of the present invention.

FIG. 8C shows a series of patches including devices in accordance with principles of the present invention.

FIG. 9A is plot of a calibration curve of patch vs. volume added for total combined area of a demonstration of a device in accordance with principles of the present invention.

FIG. 9B is a plot of a calibration curve for optical reflection of a demonstration of a device in accordance with principles of the present invention.

FIG. 10A is a cross-sectional view of a device in accordance with principles of the present invention.

FIG. 10B are photographs of a device in accordance with principles of the present invention.

FIG. 10C is a plot of data collected from a demonstration of a device in accordance with principles of the present invention.

### DEFINITIONS

As used herein, “swellable component” means any material or component that physically increases its total dimensions and volume as it absorbs sweat, or another fluid, as specified by the present invention.

As used herein, “defined collection area” means the area adjacent to skin, or the collection surface, from which sweat, or another fluid, is collected in a manner that does not have significant interference from sweat or fluid from areas outside of the defined collection area.

### DETAILED DESCRIPTION OF THE INVENTION

Embodiments of the disclosed invention are directed to osmotic draw systems with specific applications taught for preconcentration systems. Certain embodiments of the disclosed invention show components and materials as simple individual elements. It is understood that many such components and materials may be multifaceted. Certain embodiments of the disclosed invention show sub-components with more sub-components still needed for use of the device in various applications, which are known (e.g., a battery, antenna, adhesive, strap, etc.), and for purposes of brevity are not explicitly shown in the diagrams or described in the embodiments of the disclosed invention.

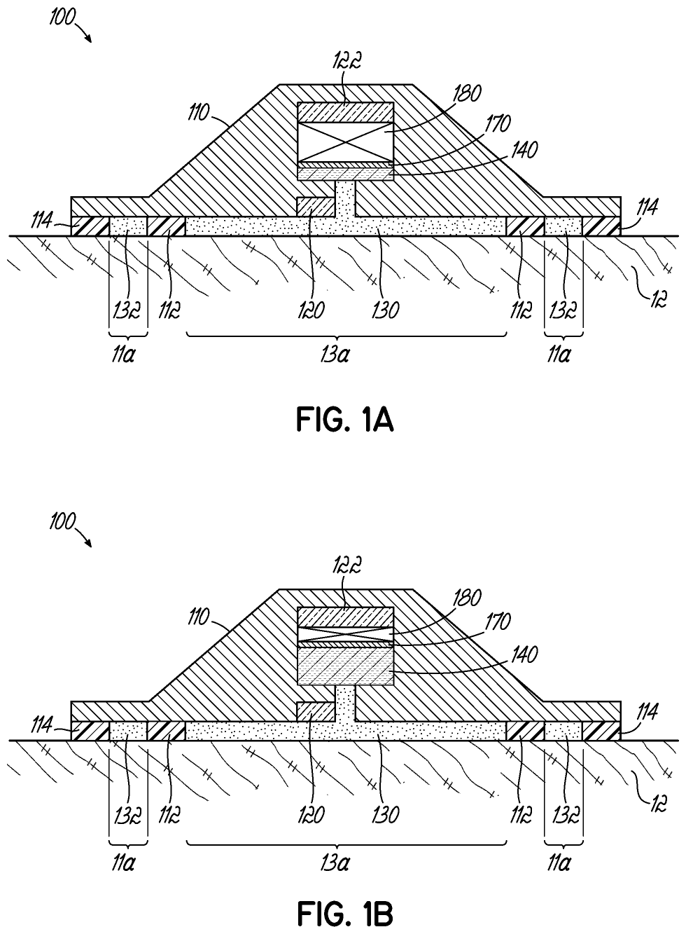

With reference to FIG. 1A, in an embodiment of the disclosed invention, a device 100 adapted to be placed on skin 12 may include a substrate or housing 110; isolation materials 112, 114; wicking or microfluidic components 130, 132; at least one sweat sensor 120; a swellable component 140; at least one sensor 122; and components 170, 180. In some embodiments, components 170,180 are optional. In various embodiments, the substrate or housing 110 could be comprised of a polymer such as PET or acrylic; the isolation materials 112, 114 could be o-rings, adhesives, petroleum jelly, or other materials; the wicking or microfluidic components 130, 132 could be microfluidic channels, paper, textiles, or other suitable materials; and the swellable component 140 could be comprised of material or multiple materials including, but not limited to, polyacrylate, polyacrylamide, starch-acrylonitrile co-polymer ethylene maleic anhydride copolymer, cross-linked carboxymethylcellulose, polyvinyl alcohol copolymers, cross-linked polyethylene oxide, or starch grafted copolymer of polyacrylonitrile.

The sensor 122 is configured to measure or detect a property or condition of swellable component 140, such as (for example) thickness, volume, dimension, optical reflectance, or the position of at least a portion of the swellable component 140. Alternatively, the sensor 122 may detect the position of optional component 170 that may be moved or otherwise affected by swellable component 140. This can be achieved by one or more techniques, including, but not limited to, having sensor 122 include a capacitive transducer, capacitive displacement sensor, eddy-current sensor, ultrasonic sensor, grating sensor, hall effect sensor, inductive non-contact position sensor, laser doppler vibrometer, linear variable differential transformer, photodiode array, piezo-electric or piezo-resistive transducer (piezo-electric), potentiometer, proximity sensor (optical), rotary encoder (angular), seismic displacement pick-up, string potentiometer (also known as string pot., string encoder, cable position transducer), confocal chromatic sensor, or other suitable sensors 122. For example, sensor 122 could be a pressure sensor, component 180 could be a spring, and component 170 could be a metal plate. Using Hook's law, the sensor 122, being a pressure sensor in this example, could detect compression of component (spring) 180, as component 180 is compressed due to swellable component 140 increasing in dimension or volume over time (see FIG. 1B, which shows the device 100 having received sweat from the skin 12 and the swellable component 140 having increased in volume and dimension).

Alternatively or additionally, as another example, sensor 122 could be an optical proximity sensor (such as a co-planar light emitting diode and a photodiode), component 170 could be a fully or partially reflective plate (such as aluminum), and component 180 could simply be air that can be vented (not show) such that the position of component (plate) 170 can be detected as it is moved due to swellable component 140 increasing in dimension or volume over time (again, see FIG. 1B, which shows the device 100 having received sweat from the skin 12 and the swellable component 140 having increased in volume and dimension). Still alternatively, black pigment or dye could be added to swellable component 140 such that Fresnel reflection would dominate the optical proximity sensor signal and no component 170 would be needed. The above-described examples for the various components and operation of the device 100 are nonlimiting, and further examples are possible. However, further examples are not explicitly described here simply for the purpose of brevity. The change in volume or dimension of swellable component 140 can then be used to calculate the sweat generation rate using math, predictive algorithms, look up tables, or other suitable methods, as will be described in the Examples section.

With reference to FIGS. 1A and 1B, several further features of the present invention are described in greater detail. The wicking or microfluidic component 130 absorbs sweat from region 13a of the skin 12, and the wicking or microfluidic component 132 absorbs sweat from regions 11a of the skin 12. The regions 11a from which the wicking or microfluidic component 132 absorbs sweat is outside of the regions 13a from which the wicking or microfluidic component 130 absorbs sweat. By absorbing sweat from regions 11a outside of skin regions 13a covered by wicking or microfluidic component 130, the wicking or microfluidic components 132 can help create a defined sweat collection area that is limited primarily to skin that is proximal to wicking or microfluidic component 130 (i.e. primarily region 13a). To assist in this, isolation materials 112, 114 prevent sweat absorbed by wicking or microfluidic component 132 from interacting with sweat absorbed by wicking or microfluidic component 130. Further, sensor 120 could serve multiple functions. For example, sensor 120 could measure the concentration of at least one analyte in sweat, such as an electrochemical aptamer sensor for cortisol. For example, sensor 120 could measure sweat conductivity, and component 170 could be an electrode in electronic communication with sensor 122, and sensor 122 may be an electrical resistance sensor. Component 170 and sensor 122, together with knowing sweat conductivity by virtue of sensor 120, are able to determine electrical resistance and therefore thickness of swellable component 140. Swellable component 140 is shown as being horizontally confined by housing 110, and may benefit from swellable component 140 being made of powder or microbeads such that swellable component 140 does not form a dome or convex shape as it absorbs sweat and swells. Alternatively, swellable component could be horizontally smaller than the size of the horizontal space of housing 110, or housing 110 could be absent from the device 100. Alternatively, doming or convex shape formation of the swellable component 140 could be accommodated for by sensor 122 in some fashion or by software, algorithms, or other suitable methods. Sensor 120 could also be a sweat flow rate sensor such as thermal mass flow sensors made by Sensiron Inc. allowing device 100 to display or combine a plurality of measurements of sweat generation rate.

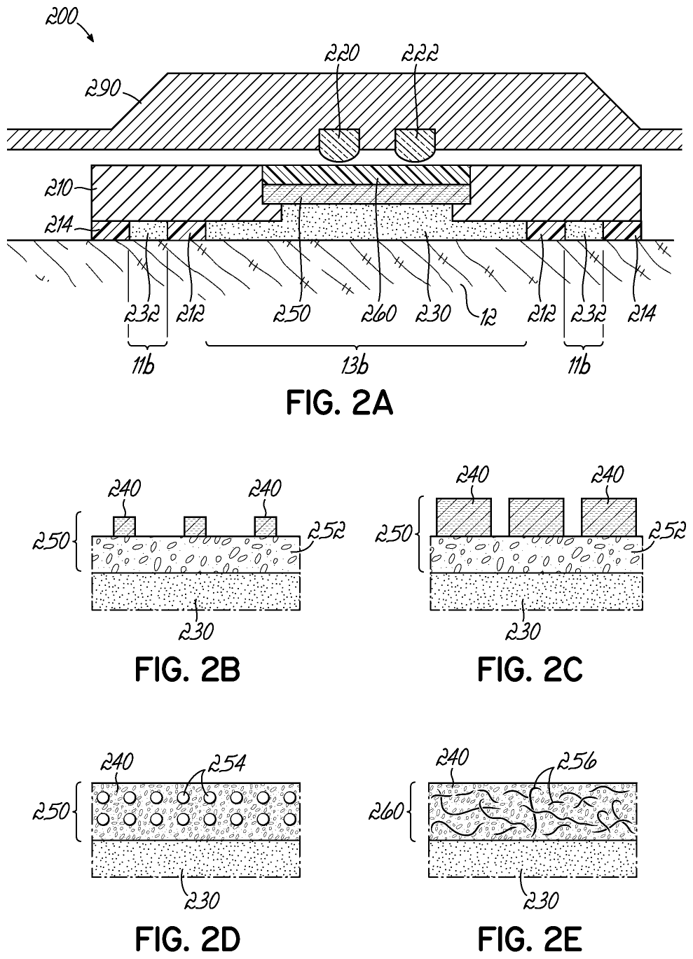

Referring now to FIG. 2A, another embodiment of a device in accordance with principles of the present invention is shown. In this embodiment, a device 200 can use other means for measuring total sweat volume or sweat rate. For example, device 200 could be a single unified device, with element 290 and sensors 220, 222 being elements with similar function to housing 110 and sensors 120,122 described previously with respect to FIG. 1A. Alternatively, one or more sensors 222, 220, and element 290 and/or other elements could form a separate, stand-alone device such as a smart watch or other wearable device product. Device 200 can include wicking or microfluidic components 230, 232 that could be microfluidic channels, paper, textiles, or other suitable materials. By absorbing sweat from regions 11b outside of skin covered by wicking or microfluidic component 230, the wicking or microfluidic components 232 can help create a defined sweat collection area 13b that is limited primarily to skin that is proximal to wicking or microfluidic component 230 (i.e. primarily region 13b). To assist in this, isolation materials 212, 214 prevent sweat absorbed by wicking or microfluidic component 232 from interacting with sweat absorbed by wicking or microfluidic component 230. Sensor 222, housing 210, and element 250, or layer 260 could be similar in function to one or more of swelling component 140 or components 170, 180.

With further reference to device 200 in FIGS. 2A-2E, which represents greater detail for element 250, an optically sensed sweat sensing device could include an element 252, such as a track etch or other sweat-porous membrane or material, that is optically reflective. For example, a track etch membrane could be coated with reflective aluminum coatings to form element 252. A swellable material 240, like that taught previously regarding swellable component 140, may contain a colourant, such as dye or pigment, such as a carbon black pigment that disperses readily in a gel or in water. The swellable material 240 could be a simple non-woven mesh or a netting. As shown in FIG. 2C, with an uptake of sweat, swellable material 240 swells and covers or optically blocks more of a surface of the element 252. As a result, the optical reflectance decreases between element 250 in FIG. 2B and element 250 in FIG. 2C and more sweat is absorbed into swellable material 240. This optical reflectance could then be measured by sensors 220, 222 as taught previously. In one embodiment, element 290 may be a smart watch where sensors 220, 222 are built-in sensors included in the smart watch for heart rate monitoring. To aid in cross-coupling light between a sensor 220 that could be a light emitting diode and sensor 222 which could be a photodiode, layer 260 could be an optical film such as an optical diffuser that horizontally spreads light as it propagates. For example, layer 260 could be a simple translucent plastic such as white-pigment dispersed in acrylic. In some embodiments, tight alignment of sensors 220, 222 and element 250, or layer 260 may not be required because element 250, or layer 260 could have large enough area that sensors 220, 222 could be in multiple positions and still properly optically measure element 250, or layer 260. Because many smart watches have removable bands, the element 290 could also include a special strap that is adapted to a standard smart watch to allow placement of the device 200 on a bicep or other position on the body, yet still rely on the electronics and communications and screen of the smart watch.

With further reference to FIGS. 2A-2E, any optical measurement is possible for the device 200 as taught, and, for example, may be a second different optical measurement that relies on a swellable material 240 that is embedded with material 254 (see FIG. 2D) such as micro or nano-spheres, forming a photonic crystal that changes its reflectance as it swells. In this way, in some examples, the swellable component 240 contains a photonic crystal.

With further reference to FIGS. 2A-2E, any sensing modality as taught previously in this application is also possible, or is adoptable using other known sensing methods that could measure a swellable material. For example, as shown in FIG. 2E, swellable material 240 could contain nano-wires or nano-tubes or other electronically conductive material 256 that causes electrical resistance of layer 260 to change as swellable material 240 changes in volume with sweat. For example, resistance of layer 260 could increase as the volume of swellable material 240 increases as it absorbs sweat. This electrical resistance change could then be measured using sensors 120, 122 as electrodes for measuring electrical resistance, such as those used in wearable devices that measure galvanic skin response, body impedance, or skin conductance.

With further reference to FIGS. 2A-2E, the materials and features outside of elements 220, 222, 290 can be disposable and low cost. Element 252 could be low-cost paper, and not all the features shown in FIGS. 2A-2E might be needed. Furthermore, material and features can be selected to work with the expected optical ranges of detection for smart watches and other devices.

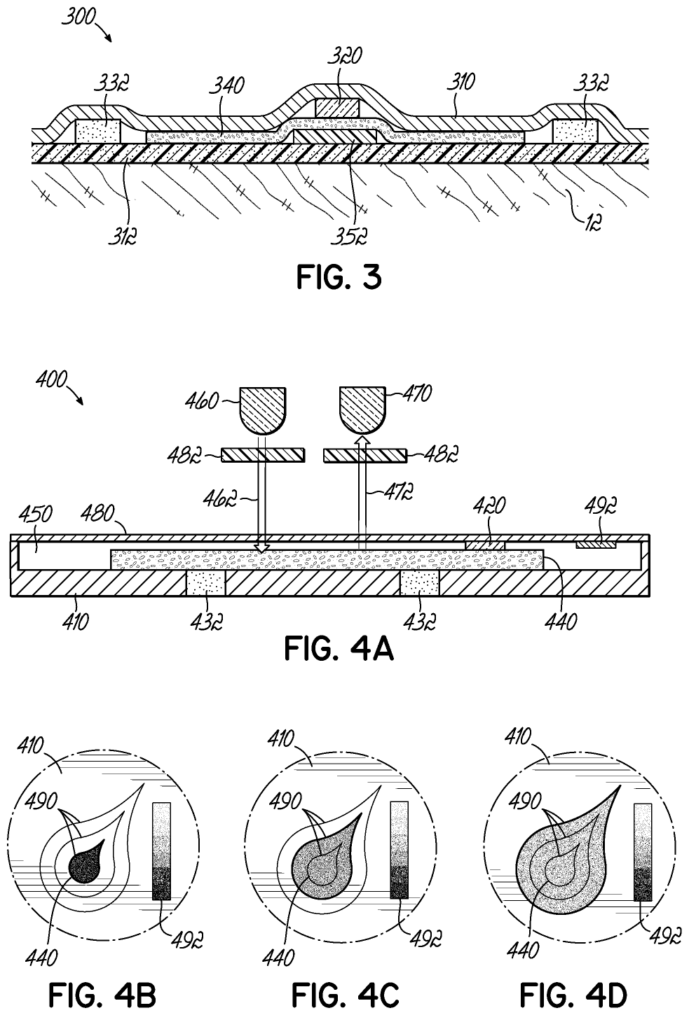

Referring now to FIG. 3, yet another embodiment in accordance with the principles of the present invention is shown. This embodiment includes a device 300 having a housing 310, which may be a plastic covering, and may include PET, and at least one sweat impermeable component 352 between a swellable component 340 and the skin 12. The swellable component 340 and or another wicking material (not shown) provides fluidic connection from the swellable component 340 to the skin surface and/or a component 312. The component 352 may be, for example, an optical reflector film such as aluminized mylar or 3M ESR reflective film. Device 300 further includes at least one material 332 such as a hydrogel such as polyacrlylate or paper that absorbs, wicks, or receives sweat such that sweat from regions outside the defined collection area does not reach the swellable component 340. Such material 332 could also be a skin adhesive, a rubber o-ring, petroleum jelly, or other suitable material that provides a defined collection area. In some examples, the component 312 may be an adhesive or a garment. For example, where petroleum jelly is used for material 332, the component 312 may be an adhesive, and may need to be removed beneath material 332 (not shown) to provide a seal against skin 12.

With further reference to FIG. 3, the device 300 may be placed on the skin 12 by being attached to the component 312, which may be a garment. The garment could include, but not be limited to, a shirt, a headband, or any other suitable material that is able to transport sweat or water vapor from the skin 12 to the swellable component 340. Alternatively or in addition, component 352 could include a transparent sweat-impermeable adhesive that adheres device 300 to the component 312. This configuration on a component 312 can improve wear or ability to view the swellable component 340.

With further reference to FIG. 3, in yet another embodiment of the present invention the device 300 includes at least one swellable component 340 that includes at least one optically responsive chemical to at least one analyte in sweat. Optically responsive chemicals may include, for example, silver chloranilate for measuring sweat chloride concentration, or a pH dye, or other colorimetric agents for analytes such as glucose, lactate, urea, etc.

It is further contemplated that embodiments, such as that shown in FIG. 3, may measure other substances present on, or generated by, the user or wearer. For example, the device 300 may be used to measure wound exudate on a wound on the skin 12. Wound exudate volume and rate of production of would exudate is a useful tool for analyzing would healing status. The devices of the present invention can therefore be incorporated into bandages or wound-protecting materials.

With further reference to FIG. 3, sensor 320 could serve multiple functions. For example, sensor 320 could measure the concentration of at least one analyte in sweat, such as an electrochemical aptamer sensor for cortisol. For example, sensor 320 could measure sweat conductivity. Swellable component 340 is shown as being horizontally confined by housing 310, and may benefit from swellable component 340 being made of powder or microbeads such that swellable component 340 does not form a dome or convex shape as it absorbs sweat and swells. Sensor 320 could also be a sweat flow rate sensor, such as thermal mass flow sensors made by Sensiron Inc., allowing device 300 to display or combine a plurality of measurements of sweat generation rate.

Referring now to FIGS. 4A, 4B, 4C, and 4D, aspects of the disclosed invention are directed to a system, device, etc. for measuring fluid exposure. Embodiments of the invention may include a device 400, the device 400 including a substrate or housing 410, at least one swellable component 440, and a fluid collector, such as wicking or microfluidic component 432 that can be in fluid communication with the swellable component 440. Various materials may be used to form the substrate or housing 410 such as PET or acrylic. The device 400 may further include a space 450 that allows the dimensions of the swellable component 440 to increase.

Further, the swellable component 440 may contain a dye or pigment, such as a carbon black pigment, that disperses readily in a gel or in water and acts as particles that absorb and/or scatter electromagnetic radiation. The swellable component 440 material could be a simple non-woven mesh or netting, and may include a mesh geometry. The swellable component 440 could include the substrate or housing 410 itself. Alternatively, the swellable component 440 could include the wicking or microfluidic component 432 itself.

As described above, the fluid collector could be a wicking or microfluidic material 432 that could include microfluidic channels, paper, textiles, or other suitable materials. The wicking or microfluidic material 432 may have various sizes and positions within the device, and in many embodiments is in fluid contact with the swellable component 440.

The device 400 itself—or a system including the device 400—may further include at least one measurement modality (e.g., for measuring the dimension of the swellable component 440 in order to calculate a criterion—such as a fluid generation rate and/or fluid volume). In certain embodiments of the present invention, this may include the use of a measuring unit (such as may be—or may be included in—a smart phone, smart watch, or a tablet), wherein that measuring unit is used in concert with device 400.

In use, with the uptake of fluid, swellable component 440 swells. As a result of the swelling, the volume of the swellable component 440 increases, the density of scatter decreases, and its optical reflectance decreases. As described above, embodiments of the device 400 may include a swellable component 440 having a dye or pigment that acts as particles that scatter electromagnetic radiation, but that disperses in water; thus, as the swellable component 440 is exposed to a fluid—and swells—the dye or pigment is dispersed, thereby decreasing the density of scatter and/or absorption provided by that dye or pigment.

The optical reflectance of the material included in the swellable component 440 can be measured by a measuring unit comprising an emitting device 460 that emits an electromagnetic radiation 462 that is subsequently reflected as reflected radiation 472 by the swellable component 440. In one aspect of the present invention, the measuring unit could be a smart phone, smart watch, or a tablet, where the emitting device 460 and a detecting device 470 are simply the already-built in devices for signal acquisition, like, for example, visible or infrared image acquisition. Reflectance measurements may be taken before, during, and/or after exposure to fluid. Additionally, the sensitivity of the measuring unit could be enhanced by implementing techniques like the lock-in method. In alternative embodiments, visual inspection of the device may be used.

As described above, once the swellable component 440 has swelled due to the uptake of fluid, optical reflectance of the swellable component can then be measured. To aid the reflectance measurement and reduce potential optical artefacts, the device 400 may further include an electromagnetic radiation diffusor 480. The role of the electromagnetic radiation diffusor 480 is to diffuse reflected radiation 472 reflected from swellable component 440. For example, electromagnetic radiation diffusor 480 could be a simple translucent plastic such as white-pigment-filled acrylic or other electromagnetic radiation diffusing material.

To further aid reflectance measurement, polarization foils 482 may be used. The polarization foils 482 are placed in front of the emitting device 460 and detecting device 470 rotated by 90°—also called a crossed polarization configuration. Alternatively, the polarization foils 482 could be placed directly on the top of the swelling material 440 or on top of the electromagnetic radiation diffusor 480.

And to further still aid reflectance measurement, calibration scales 492 can be used. The goal of the calibration scale 492 is to allow for calibration of the device 400.

The change in reflectance of the swellable component 440 that is measured by the measuring unit can then be used to calculate the fluid exposure using math, predictive algorithms, look up tables, or other suitable methods, as will be explained in greater detail in the Examples section.

Further still, when the volume of swellable component 440 increases, this volume increase could be measured using a sensor 420. This sensor 420 could be, but is not limited to, a capacitive transducer, capacitive displacement sensor, eddy-current sensor, ultrasonic sensor, grating sensor, hall effect sensor, inductive non-contact position sensor, laser doppler vibrometer, linear variable differential transformer, photodiode array, piezo-electric or piezo-resistive transducer (piezo-electric), potentiometer, proximity sensor (optical), rotary encoder (angular), seismic displacement pick-up, string potentiometer (also known as string pot., string encoder, cable position transducer), confocal chromatic sensor, or other suitable sensors. For example, in an embodiment of the present invention sensor 420 could be a pressure sensor.

FIGS. 4B-4D illustrate a top-down view of the device 400 shown in FIG. 4A. With an uptake of fluid, swellable component 440 swells and expands and eventually fills a part or the totality of space 450. As a result of the swelling, the surface area of the swellable component 440 expands. The surface area of the component 440 can be measured optically by the different embodiments of the measuring unit described above. To aid in the measurement, markers 490 (see FIGS. 4B, 4C, and 4D), that help measuring the surface area of a swellable component 440 could be used.

To aid the measurement of the surface area, an asymmetrical shaped swellable component 440 may be used (e.g., the teardrop shape shown in FIGS. 4B-4D is merely one example of an asymmetrical shape that may be used). In some examples, the swellable component 440 includes an asymmetrical geometry. The asymmetries of the material may help a reader in reading the sensor, as such a shape could help potential image processing algorithms like, for example, movement compensation, inhomogeneous illumination, angle correction, etc. Such algorithms can be much easier performed if the shape if the swellable component is asymmetrical. In some examples, the swellable component 440 may be one of a plurality of swellable components 440 with independent geometries for the swellable components 440. In some examples, at least some of the plurality of swellable components 440 have geometries different from other swellable components 440 included in the device 400.

The change in volume of swellable component 440 can be used to calculate the internal and external fluid exposure using math, predictive algorithms, or look up tables, or other suitable methods, as will be taught in the Examples section.

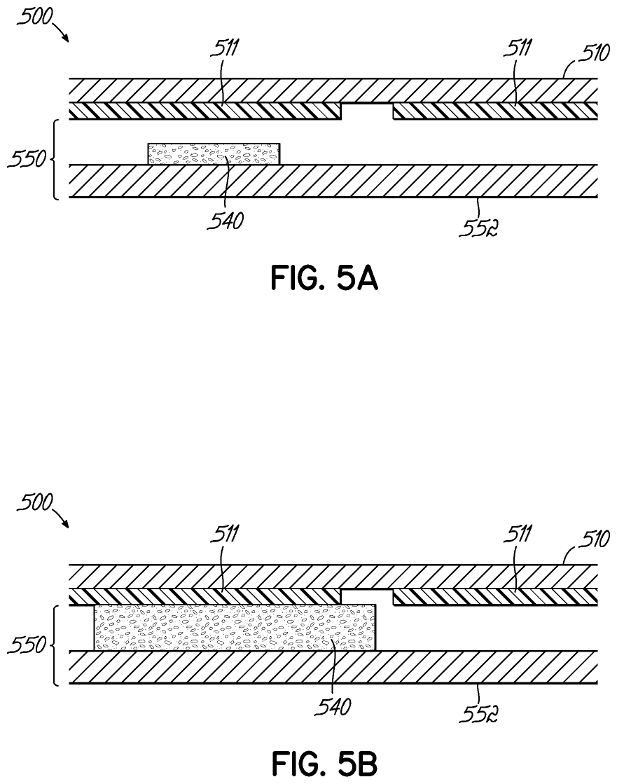

Referring now to FIGS. 5A and 5B, yet another embodiment in accordance with principles of the present invention is shown. In this embodiment, a portion of a device 500 includes a substrate or housing 510 that could be comprised of a polymer such as PET or acrylic, and at least one optically opaque material 511, such as a black or white film of PET plastic. A swellable component 540 is shown as being located on element 552, and swellable component 540 may be made of powder or microbeads such that swellable component 540 does not form a dome or convex shape as it absorbs sweat and swells. As shown in FIG. 5A the swellable component 540 is initially not visible due to material 511, and as shown in FIG. 5B has absorbed sweat and has become visible or measurable through a portion of the device not covered by optically opaque material 511. In such a configuration the device can provide a simple threshold determination of a sweat volume. A plurality of openings or holes (not shown) in an optically opaque material 511 could also be used to provide multiple indications of the amount of sweat absorbed by at least one swellable component 540.

With further reference to FIGS. 5A and 5B, element 552, such as a track etch or other sweat-porous membrane or material, is provided that is optically reflective. For example, a track etch membrane could be coated with reflective aluminum coatings to form element 552. As shown in FIG. 5B, with an uptake of sweat, swellable component 540 swells and covers or optically blocks more of a surface of the element 552. As a result, the optical reflectance decreases between element 552 in FIG. 5A and element 552 in FIG. 5B and more sweat is absorbed into swellable component 540. An element 550 includes the swellable component 540 and the element 552.

The following describes a potential implementation of a fluid exposure device designed to measure sweat content and sweat rate:

A sweat sensing device with a defined sweat collection area of 9 cm² could be placed on the forearm with a gland density of 100 glands/cm² and a sweat generation rate of 5 nL/min/gland. The swellable component could be horizontally confined and have an initial cross-sectional area of 1 cm² and an initial thickness of 0.127 cm.

Therefore, the sweat delivered to the swellable component could be delivered at a rate of 4.5 μL/min. The swellable material could increase its cross-sectional area at a rate of 0.00625 cm²/μL. Therefore, after 1 hour the swellable component would increase in cross-sectional area by approximately 1.7 cm². This increase in the surface area can be measured by the means described precedingly and the obtained value can be used to calculate the amount of generated sweat, in this particular case 270 μL, and the sweat rate, in this case 4.5 μL/min. For more accuracy, the volume of sweat in the collection area should be subtracted from the amount of generated sweat.

A remaining concern might be the linear behavior of the swellable device for monitoring use for different amount of time and for various sweat rates. Indeed, the swellable material may exhibit a linear increase of its volume when absorbing sweat until a certain amount of absorbed sweat where saturation effects occurs. Similarly, the swellable material may not behave linearly with very low amount of sweat. The properties of the swellable material, as well as its initial thickness and initial cross-sectional area, as well as the surface of the sweat collection area can be designed and adjusted so that the sensing device can accurately measure sweat content and sweat rate for a desired amount of time and for different sweat rates.

### Example

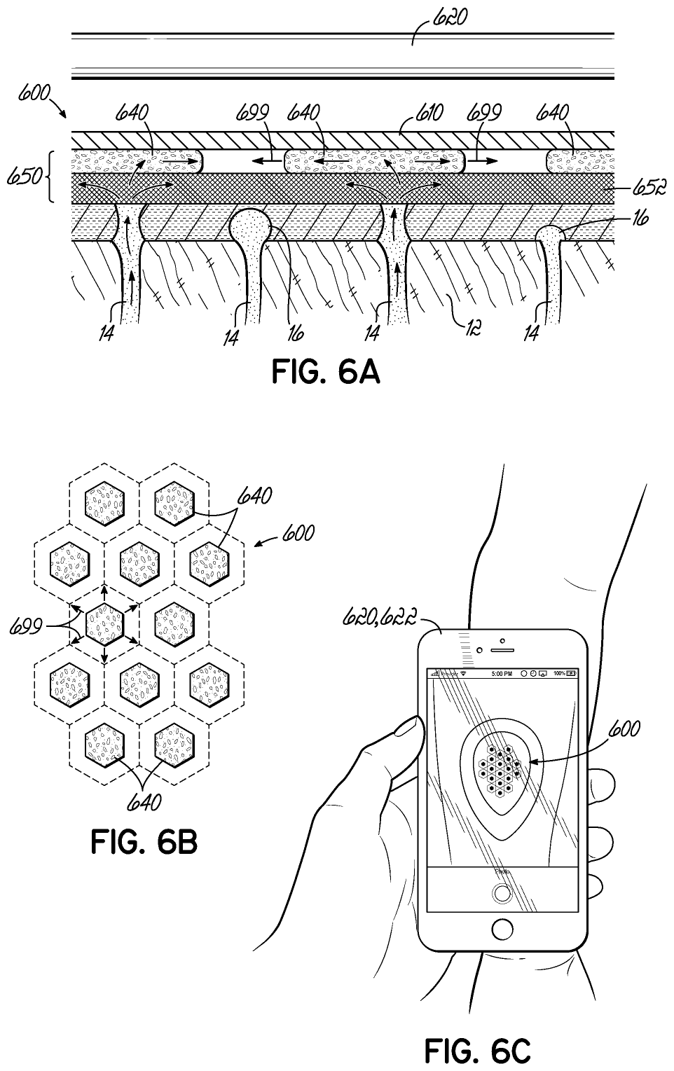

With further reference to FIGS. 6A-10C, the following Example supports aspects and embodiments of the present invention and teaches additional aspects and embodiments of the present invention.

Materials

Samples of adhesive materials 3M9964, 3M4076 were obtained from 3M (Maplewood, Minn.). 99.0% Sodium chloride (NaCl), 99% poly (yin alcohol), 93% sodium hydroxide and 37% hydrochloric acid were obtained from Sigma-Aldrich (St. Louis, Mo.). Super absorbent polymer (ST-250*) was obtained from Newstone (Tokyo, Japan). Blue colorant (450C) were obtained from Cabot (Alpharetta, Ga.). Plastic sheets (30 cm×30 cm, 0.5 mm thick) were obtained from Grafix (Maple Heights, Ohio). The Rayon textile were obtained from WPT Nonwovens (Beaver Dam, Ky.). Ultrapure water (resistivity: 18.2 MS2 cm) was obtained from an EMD Millipore Direct-Q® 3 UV water purification system (Darmstadt, Germany)

Swellable Component Hydrogel Fabrication

A swellable component 640 (FIGS. 6A and 6B) of a super absorbent polyacrylamide gel was prepared by adding 10 g of super absorbent polymer to 400 mL of water and stirring for 1 hour at 60° C. The mixture was dried in oven at 60° C. overnight and lost ˜200 g of water creating a gel-like material (a super absorbent gel). 10% of PVOH was added to the super absorbent gel along with 0.5 mL of the Cabot 450C blue colourant. This mixture was then doctor-blade coated onto a plastic sheet and dried at 40° C. overnight. The resulting hydrogel film was then stored along with a desiccant to prevent water absorption during storage. The hydrogel films were then laser cut into swellable component hexagons 640 using a Desktop laser Platforms (VLS3.50, Scottsdale, Ariz.) at settings of 25% of power and 100% of speed.

Patch Fabrication

Simple layer-by-layer lamination of the device was utilized.

In Vitro Testing

The photographs of the characterization were taken by using a Single Lens Reflex Camera from Canon U.S.A., Inc (Melville, N.Y.). The hexagon 640 area change was coordinately analysed by using two software programs, Photoshop (available at adobe.com) for isolating the colour and Image-J (available at imagej.net) for calculating the total area of all the hydrogel hexagons 640 in a patch.

Patch Construction

Referring to FIGS. 6A-10C, the basic wearable patch device 600 contains an element 650 including an element 652, the element 652 may be a textile nano-channel wicking layer, a coloured hydrogel as a swellable component 640 that swells at least in the dimension indicated by arrows 699, and housing 610, which may be a clear upper film, to prevent evaporation of water and/or contamination of the hydrogel 640 from external water/sweat. The housing 610 may also include an anti-fogging coating or other suitable material or mechanism to prevent water condensation from hindering optical measurement of the swellable component 640. The element 652, which may be a textile nano-channel wicking layer as described above, serves to provide a wicking contact to the entire collection area on skin 12 and collect sweat 16 from sweat ducts 14, and also provides white reflective background to contrast against the coloured swellable component 640. Rayon was utilized for the element (textile nano-channel wicking layer) 652 with 6.80 cm² total area, 0.64 mm thickness, and 4.02 cm² contact with skin. Rayon was chosen because it wicks sweat along nano-grooves in the Rayon such that the Rayon layer need not become saturated (full of sweat) before it can transport sweat from the skin to the swellable components 640. The total fluid volume of the Rayon is therefore at most ˜6% of the thickness of the Rayon. At a thickness of 0.64 mm, the Rayon creates a dead-volume in the patch that is ˜4 μL/cm² or less. Onto the element 652 (textile nano-channel wicking layer), hexagons of blue-coloured swellable component 640, in this Example a hydrogel, are placed with geometry of 2.8 mm diameter and 50-130 μm thickness. The hexagons 640 occupy 23.6% of the total Rayon collection area. As the swellable component(s) 640 absorb sweat they increase in geometry as indicated by arrows 699, which can then be measured by taking a photo of the hexagon using device 620, 622 (shown in FIG. 6C) and using software to analyze the increased size as the coloured swellable component 640 covers more area of the white-coloured element 652, which may be a textile nano-channel wicking layer (shown in FIG. 6A).

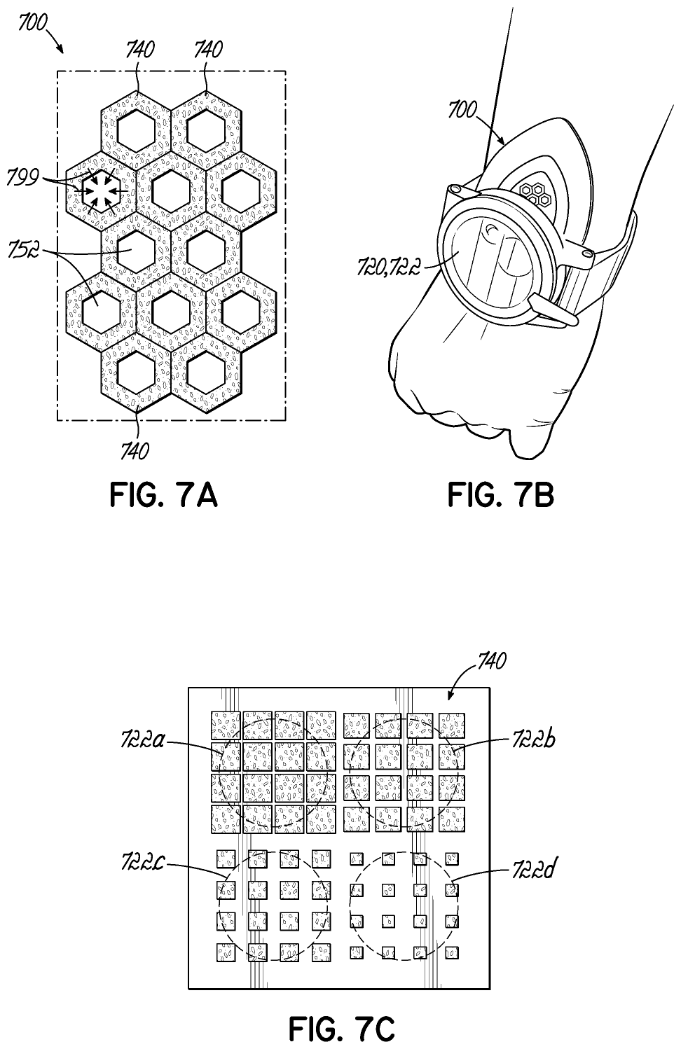

Alternatively, in a similar device shown in FIGS. 7A-7C, a basic wearable patch device 700 contains a coloured hydrogel as a swellable component 740 that swells at least in the dimension indicated by arrows 799. The swellable component 740 are also kept separated by a surrounding lattice of double-sided adhesive (not shown) so they can never overlap each other. As the swellable component(s) 740 absorb sweat they increase in geometry as indicated by arrows 799, which can then be measured by using a device 720, 722 (shown in FIG. 7B) for measuring the decreased optical reflectance of non-blue light as the blue swellable component 740 covers more area of the white-coloured element 752, which may be a textile nano-channel wicking layer.

The thickness of the swellable component 640, 740 of the embodiments of FIGS. 6A-6C and 7A-7C, a hydrogel material in this example, can be adjusted to the amount of sweat that needs to be absorbed. A thinner hydrogel would allow the device to be more sensitive to smaller volumes of sweat, but if the hydrogel is too thin, then: (1) the device could have limited usage time before the hydrogel reaches its swelling capacity; (2) the hydrogel could be too transparent to optically measure; (3) the hydrogel could become very flimsy (mechanically weak) such that horizontal expansion of the hydrogel could be un-reliable (edge-pinning, friction, wrinkling, etc.). As illustrated in FIG. 7C, a plurality of swellable components 740 geometries, in this example hydrogel geometries, (thickness, optically measureable area, etc.) and plurality of measurement locations or sensors 722a, 722b, 722c, 722d is also possible. For example, measurement location 722d could respond more quickly than measurement location 722a in reduced optical reflectance with absorption of sweat. Similarly, location 722a would respond more slowly (require more sweat volume to change significantly in optical reflectance) than measurement location 722d.

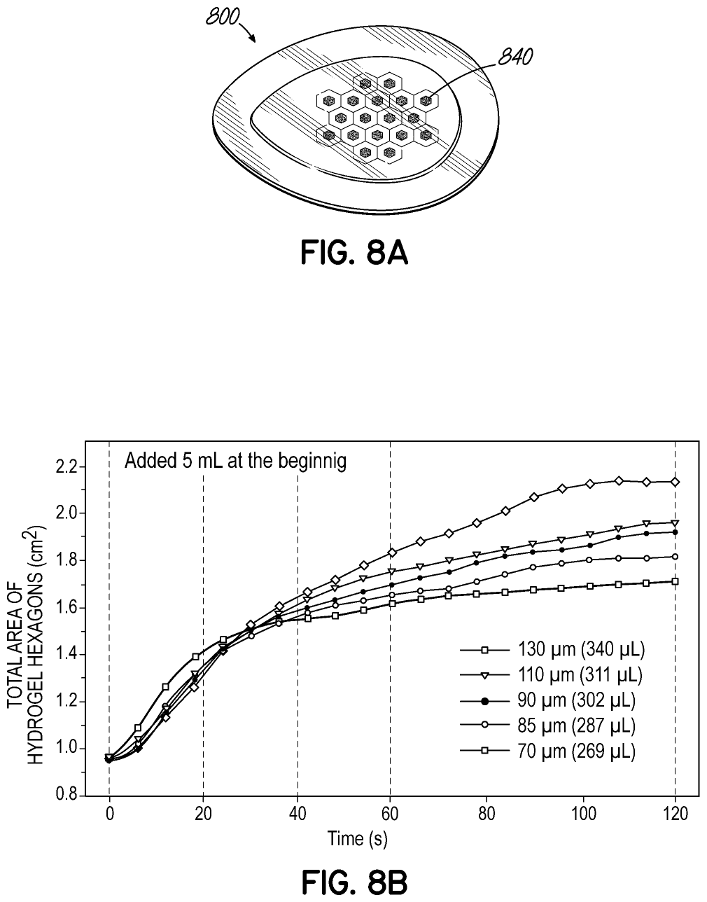

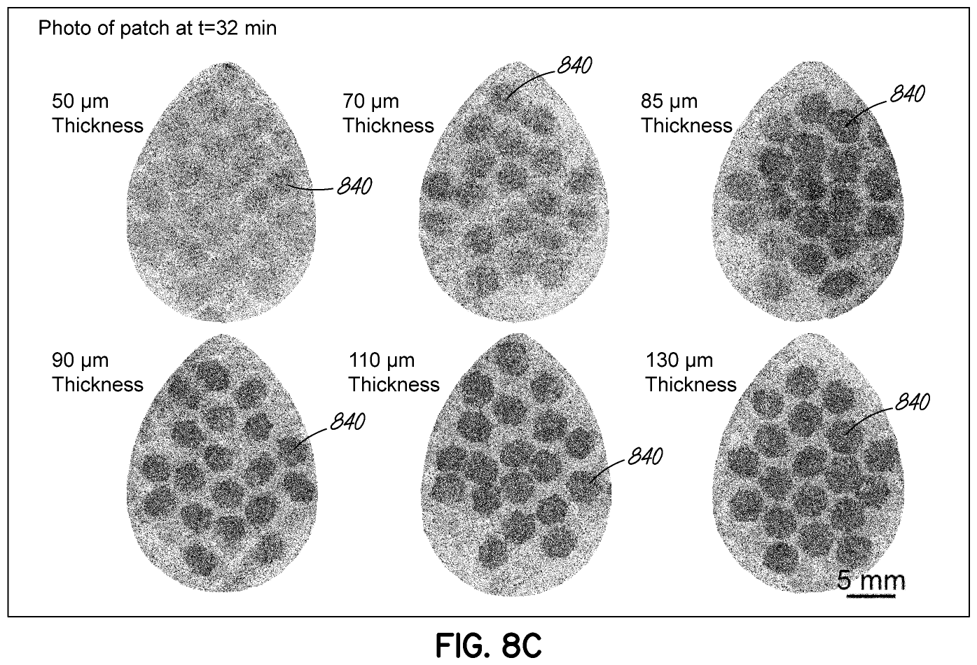

FIG. 8A shows an example of a fully assembled device 800 with swellable components 840, which may be a hydrogel. FIG. 8B demonstrates how varying the initial dry swellable component 840 thickness (70 μm, 85 μm, 90 μm, 110 μm, and 130 μm) impacts the rate and maximum volume of fluid absorption. The data in FIG. 8B was measured by analysing the total combined visible area of the swellable components 840, each being hexagonal, in each patch. The swellable components 840 may need a minimal thickness, for example, a swellable component 840 made of hydrogel having a thickness of 50 μm is not shown because at 50 μm thick the hydrogel was too optically transparent and too mechanically flimsy. 5 mL of 1 mM NaCl solution was added all at once to the patch, which exceeds the fluid capacity of the patch, such that measurements could be made at the maximum speed of swelling and the maximum swelling capacity. At the beginning, no significant variations can be noticed between the different thicknesses and all swellable components 840 expand at a similar rate (˜0.1-0.2 mm²/s). After approximately 40 seconds, the thinner hydrogels tend to quickly plateau in growth rate (0.20 mm²/s, 0.31 mm²/s, 0.41 mm²/s, 0.42 mm²/s and 0.60 mm²/s for 70 μm, 85 μm, 90 μm, 110 μm, and 130 μm respectively). It is currently speculated that the onset of saturation (plateau) of growth rate of the hydrogel is an inherent limitation of the hydrogels as fabricated in this work, and/or potentially caused by reduced mechanical strength of the hydrogel as it swells with water. The custom-made polyacrylamide hydrogels in this work swell by ˜20-30× in <2 minutes. Polyacrylamide hydrogels are known to swell up to 350×-650× of their initial volume, but complete swelling is a longer process (hours or more) and involves weaker swelling force as the hydrogel absorbs more and more water.

FIG. 8C presents a series of patches taken after 32 minutes for different hydrogel thickness. At such a time, the observable increase in surface area is slowing and/or saturating. As expected, the thicker the material, the larger the surface increase due to improved mechanical strength of the hydrogel film as it expands (less wrinkling). The colour of the patch is also visibly affected by the hydrogel thickness with the 130 μm sample exhibiting the darkest colour. Based on the results of FIG. 8C, a 85 μm initial dry hydrogel thickness was chosen for the remainder of the experiments reported in this Example.

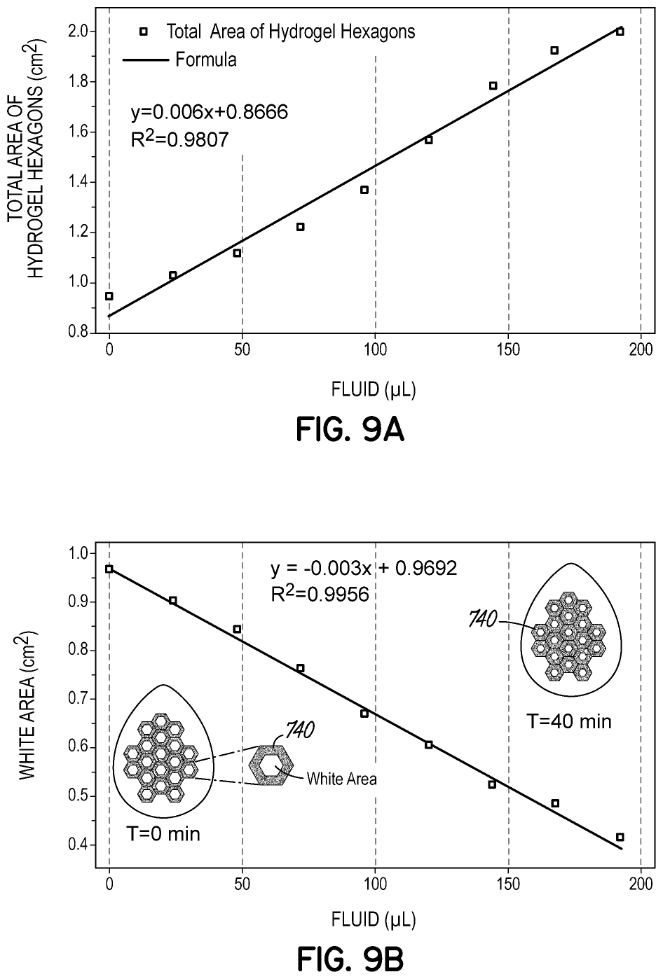

Next, as shown in FIG. 9A a calibration curve was created for total combined area of the 19 hydrogel hexagons in each patch vs. volume added. Fluid volume was increased at 4.8 μL/min over 40 minutes for a total final volume of 192 μL. Because the data confirmed a rapid swelling response for the hydrogels (approximately few minutes), it can be assumed that the hydrogel swelling is complete. For a total sweat collection area of ˜4 cm², 4.8 μL/min would represent a sweat generation rate of ˜1.2 μL/min/cm² and for 100 to 150 active glands/cm² a glandular sweat generation rate of 12 to 8 nL/min/gland. This confirmed that the patch design would be properly designed for in vivo validation experiments. The calibration data also showed a linear response to sweat volume, which is attractive from a measurement perspective, but was unexpected from a hydrogel swelling perspective. Fluid addition directly translates to increased hydrogel volume (V), but if the hydrogel were to uniformly swell in all directions, hydrogel volume (V) would translate to increased visible area (A) for the hydrogel with a mathematical ⅔^{rd} root dependence (non-linear). It is initially speculated that friction in the patch could hinder lateral spreading of the hydrogel, but even when testing freely floating hydrogels it was observed that they increase in thickness at a rate faster than they increase in horizontal dimension.

As shown in FIG. 9B, calibration was also performed for optical reflection. In this mode, a sub-set of the total hydrogel grid area would be measured using a simple LED and photodiode configuration similar to that already commonly deployed in smart-watches and other optical-mode wearables. Almost an identical calibration behaviour is found between the FIGS. 9A and 9B, suggesting that other alternative geometrical designs of the hydrogel are also feasible. The white area plotted in FIG. 9B is easily translatable to percent reflection measured by an optical wearable device, and importantly, the because of device intrinsically has zero-sweat calibration, change in reflection is all that needs to be measured (not absolute quantified reflection).

Before in vivo validation was to be explored, one more in vitro experiment was performed. Although polyacrylamide hydrogels should perform over a wide pH and salinity range, that assumption was tested over non-pathological ranges for, sweat pH (4 to 9) and salinity (10 to 100 mM NaCl). The data confirmed robust and repeatable performance of the hydrogels with varying pH and salinity. It is speculated that pH=4 is a lower limit for the hydrogel, because the pKₐ of carboxylic acid containing in the hydrogel copolymer is ˜4.5, and carboxyl groups of hydrogels tend to dissociate at a pH>4, such that at lower pH there is less osmotic pressure to drive the hydrogel swelling.

In Vivo Patch Design and Results

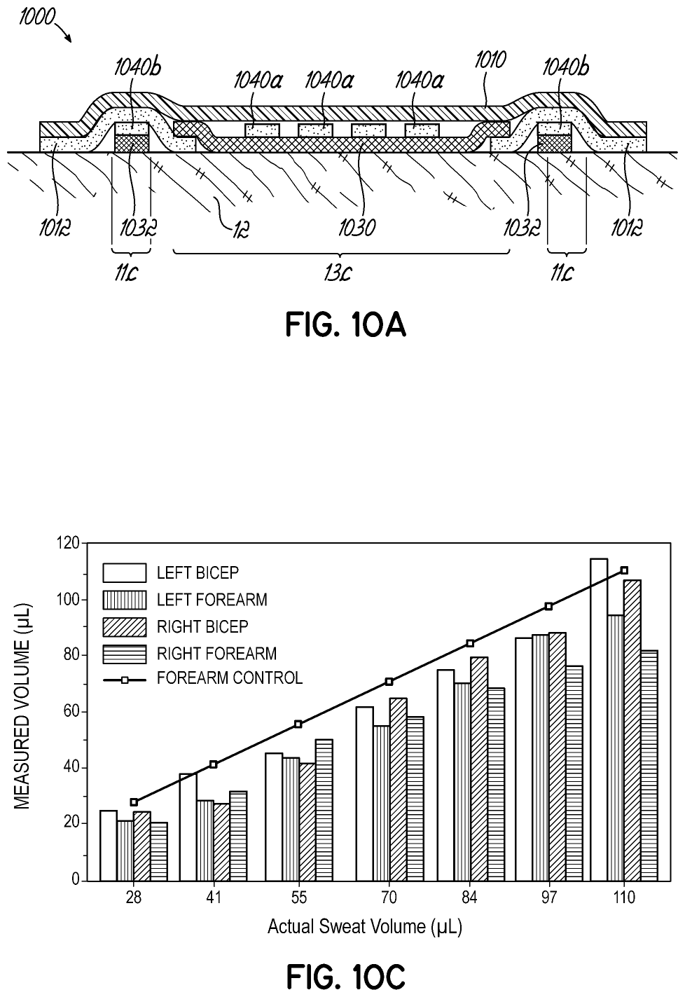

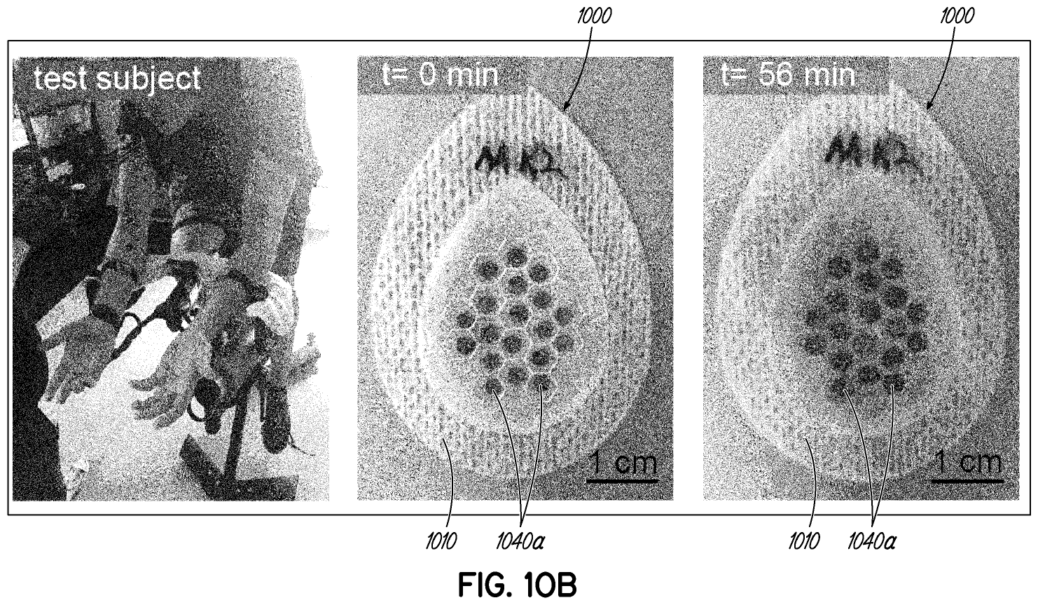

Referring to FIG. 10A, an in vivo patch device 1000 is shown. The design for the in vivo patch device 1000 validation also utilized the 85 μm thick swellable components 1040a, including hexagons of hydrogels, placed onto a textile wicking layer 1052a. The patch device 1000 includes a substrate or housing 1010 that could be comprised of a polymer such as PET or acrylic. The complete patch design is shown in FIG. 10A and also involves several additional features that are not captured in the simple diagram of FIG. 6A. Device 1000 can include wicking or microfluidic components 1030, 1032 that could be microfluidic channels, paper, textiles, or other suitable materials. By absorbing sweat from regions 11c outside of skin covered by wicking or microfluidic component 1030, the wicking or microfluidic components 1032 can help create a defined sweat collection area 13c that is limited primarily to skin that is proximal to wicking or microfluidic component 1030 (i.e. primarily region 13c). To assist in this, isolation materials 1012 prevent sweat absorbed by wicking or microfluidic component 1032 from interacting with sweat absorbed by wicking or microfluidic component 1030. To ensure a defined collection area 13c under the device 1000, the wicking or microfluidic component 1012 may be in fluid communication with a second swellable component 1040b. The wicking or microfluidic component 1012 together with the second swellable component 1040b may be a hydrogel/rayon seal material or seal ring to prevent collection of sweat from outside the defined collection area 13c (as shown in FIG. 5a). In an embodiment, the hydrogel used in the seal ring was also 85 μm thick, was 1.5 mm wide, with a perimeter length of 6.27 cm.

The in vivo validation was performed using aerobic cycling of a subject in a room held at 25° C. (see FIG. 10B). Patches were placed on both the left and right biceps and forearms of a subject (labelled as Left bicep, Left forearm, Right bicep, Right forearm). The in vivo data is shown in FIG. 10C, and includes a data set for ‘actual sweat volume’ which was measured gravimetrically near the patch (weight before and after absorption of sweat). The measured data is created using the calibration curve from FIG. 9A, and is fairly consistent for both arms. The measured data is somewhat initially consistently below that of the actual measured sweat volume. This lower initial measurement could be due to the initial volume capacity of the skin surface and the Rayon, which must be partially filled before sweat can reach the hydrogel hexagons. Overall, the in vivo data provides results that are expected based on the performance predicted by the in vitro data.

Device Calibration

Ideal use of the patches with calibration could be as follows. A user would measure nude body weight, then multiple patches applied across the body, then sweating would be generated by thermal or exercise methods. After the sweating, the multiple patches would be measured at multiple body sites by smart-phone photos and image analysis, along with a final recording of nude body weight. Then calibration curves would be generated for the individual. Once an individual was calibrated, they could then transition to one or fewer patches along with continuous optical read-out devices. Alternatively, it could also be that a user enters calibration data, for example, as a digital log-book of bodily sweat volume loss (nude-weight before and after) over time with repeated use of the device, and the device measurement software self-calibrates over time with improving predictive accuracy after each use.

The embodiments of the present invention recited herein are intended to be merely exemplary and those skilled in the art will be able to make numerous variations and modifications to it without departing from the spirit of the present invention. Notwithstanding the above, certain variations and modifications, while producing less than optimal results, may still produce satisfactory results. All such variations and modifications are intended to be within the scope of the present invention as defined by the claims appended hereto.

1. A sweat sensing device comprising:
at least one swellable component;
a defined sweat collection area in fluid communication with the swellable component;
at least one first sensor configured to directly or indirectly measure a dimension of the swellable component such that a sweat generation rate and/or a sweat volume can be calculated from the measure of dimension of the swellable component and the defined sweat collection area.

2. The device of claim 1 wherein the first sensor includes a measurement modality that is at least one of: mechanical, optical, electrical, thermal, chemical.

3. The device of claim 1, further comprising at least one microfluidic component.

4. The device of claim 3, wherein the microfluidic component couples the defined sweat collection area to the swellable component.

5. The device of claim 3, wherein the microfluidic component isolates the defined sweat collection area from sweat or fluid that is generated external to the defined sweat collection area.

6. The device of claim 1, further comprising at least one sweat-porous material.

7. The device of claim 6, wherein the sweat-porous material is optically reflective.

8. The device of claim 1, wherein the swellable component contains at least one colourant.

9. The device of claim 1, wherein the swellable component has a mesh geometry.

10. The device of claim 1, where the sensor is a smart watch.

11. The device of claim 1, further comprising at least one optical diffuser.

12. The device of claim 1, wherein the swellable component contains a photonic crystal.

13. The device of claim 1, wherein the swellable component contains an electronically conductive material.

14. The device of claim 1, further comprising at least on sweat impermeable material between the swellable component and the defined sweat collection area.

15. The device of claim 1, further comprising at least one additional material or component that provides a defined sweat collection material.

16. The device of claim 1, further comprising at least one analyte that is optically responsive to at least one analyte in sweat.

17. The device of claim 1, further comprising at least one calibration feature.

18. The device of claim 1, where the swellable component has an asymmetrical geometry.

19. The device of claim 1, further comprising at least one optically opaque material that covers at least a portion of the swellable component.

20. The device of claim 1, further comprising at least one anti-fogging material.

21. The device of claim 1, further comprising a plurality of swellable components with different geometries for the swellable components.

22. A wound exudate sensing device comprising:
at least one swellable component;
a defined wound exudate collection area in fluid communication with the swellable component;
at least one first sensor for directly or indirectly measuring the dimension of the swellable component such that would exudate generation rate and/or wound exudate volume can be calculated from the measure of dimension of the swellable component and the defined wound exudate collection area.

23. A method comprising:
absorbing an amount of sweat from skin into a swellable component in a device, the device comprising:
the swellable component;
a defined sweat collection area in fluid communication with the swellable component; and
at least one sensor; and

24. The method of claim 23, wherein the device further comprises an element having an optical reflectance, and wherein the optical reflectance is the dimension.

25. The method of claim 23, wherein the swellable component comprises an electronically conductive material having an electrical resistance, and wherein the electrical resistance is the dimension.

26. The method of claim 23, wherein the device is one of a plurality of devices, the method further comprising:
absorbing at least a portion of the amount of sweat into each of the plurality of devices;
measuring the dimension in each of the devices; and
generating a calibration curve of the dimension based on the dimension.

27. The method of claim 23, the method further comprising:
entering a calibration data into the device after repeated use of the device; and
self-calibrating the device.

28. The method of claim 23, further comprising disposing of the device.
